(19) United States
(12) Patent Application Publication
(10) Pub. No.: US 2002/0013307 A1
(43) Pub. Date: Jan. 31, 2002

(54) Use of vasopeptidase inhibitors to treat or slow the progression of cognitive dysfunction and to treat and/or prevent dementia

(76) Inventors: Pablo Lapuerta, Waterloo (BE); Michael Gold, Newtown, PA (US)

Correspondence Address: MARLA J MATHIAS, BRISTOL-MYERS SQUIBB COMPANY, PATENT DEPARTMENT, P O BOX 4000, PRINCETON, NJ, 08543-4000, US

(21) Appl. No.: 09/859,614

(22) Filed: May 17, 2001

Related U.S. Application Data
(60) Provisional application No. 60/206,342, filed on May 23, 2000.

(51) Int. Cl. A61K 31/553; A61K 31/554

(52) U.S. Cl. 514/211.05

(57) ABSTRACT

Vasopeptidase inhibitors, especially omapatrilat, are useful in treating and/or slowing the progression of cognitive dysfunction and treating and/or preventing dementia of the Alzheimer's type and vascular dementia. The vasopeptidase inhibitor may be used in combination with other pharmaceutically active agents.

Priority is claimed from application Ser. No. 60/206,342 filed May 23, 2000.

### BACKGROUND OF THE INVENTION

Over the last several years compounds have been reported in the patent and technical literature as possessing in a single molecule both angiotensin converting enzyme (ACE) inhibitory activity and neutral endopeptidase (EC24.11; NEP) inhibition activity. These compounds are of interest as cardiovascular agents particularly in the treatment of hypertension, congestive heart failure, and renal disease. These compounds are also referred to as vasopeptidase, dual metalloprotease, NEP/ACE, or ACE/NEP inhibitors.

Omapatrilat is such a vasopeptidase inhibitor which is currently undergoing clinical evaluation. Omapatrilat has the chemical name [4S-[4α(R*),7α, 10αβ]]-octahydro-4-[(2-mercapto-1-oxo-3-phenylpropyl)amino]-5-oxo-7H-pyrido[2,1-b][1,3]thiazepine-7-carboxylic acid and the structural formula

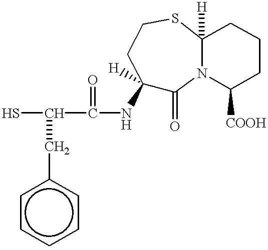

Omapatrilat, its preparation, and its use in treating cardiovascular diseases are disclosed by Robl in U.S. Pat. No. 5,508,272.

Gemopatrilat, also known in the literature as BMS 189,921, is another vasopeptidase inhibitor which is currently undergoing clinical evaluation. Gemopatrilat has the chemical name [S-(R*,R*)]-hexahydro-6-[(2-mercapto-1-oxo-3-phenylpropyl)amino]-2,2-dimethyl-7-oxo-1H-azepine-1-acetic acid and the structural formula

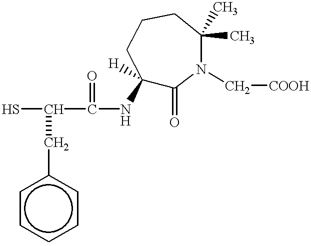

Gemopatrilat, its preparation, and its use in treating cardiovascular diseases are disclosed by Karanewsky et al. in U.S. Pat. No. 5,552,397.

Sudilovsky in U.S. Pat. No. 5,015,633 discloses the use of phosphonate substituted amino or imino acid ACE inhibitors to inhibit the loss of cognitive function and to treat or delay the progression of Alzheimer's disease or other types of dementia and/or memory disorders.

Costall et al. in U.S. Pat. No. 5,098,889 disclose the use of ACE inhibitors in combination with a drug that interacts with serotonin receptors to inhibit the loss of cognitive function.

Smith et al. U.S. Pat. Nos. 6,008,221 and 6,127,370 discloses treating occlusive vascular disease or Alzheimer's disease in a patient having elevated blood levels of homocysteine by administering folic acid, a folate, or a derivative thereof and optionally two or more agents selected fom vitamin B12, an organic nitrate or dinitrate, an ACE inhibitor, an angiotensin II antagonists, or a vasopeptidase inhibitor.

Wyss et al., “Neurologic Consequences Of Hypertension And Antihypertensive Drug Therapy”, Current Opinion in Nephrology and Hypertension, 1994, Vol. 3, p. 228-235, report that ACE inhibitors as a class most consistently lead to cognitive improvement in the overall hypertensive population.

Starr et al., “Blood Pressure and Cognitive Function In Healthy Old People”, J. Am. Geriatr. Soc., Vol. 41, p. 753-756 (1993), discuss the association between high blood pressure and cognitive impairment in healthy, drug-free, older population.

### SUMMARY OF THE INVENTION

This invention is directed to the use of a vasopeptidase inhibitor to treat and/or slow the progression of cognitive dysfunction and to treat and/or prevent dementia including senile dementia of the Alzheimer's type and vascular dementia. Preferred vasopeptidase inhibitors for these uses are omapatrilat or a pharmaceutically acceptable salt thereof, or the combination of omapatrilat or a pharmaceutically acceptable salt thereof and a diuretic, preferably hydrochlorothiazide, or gemopatrilat or a pharmaceutically acceptable salt thereof, or the combination of gemopatrilat or a pharmaceutically acceptable salt thereof and a diuretic, preferably hydrochlorothiazide, or mixtures thereof. Most preferred is the use of omapatrilat.

One or more vasopeptidase inhibitors can be employed individually or in combination to treat and/or slow the progression of cognitive dysfunction and to treat and/or prevent dementia according to this invention. The vasopeptidase inhibitor or inhibitors can also be employed in combination with one or more pharmaceutically active agents known to be useful in the treatment of cognitive dysfunction and/or dementia. The combination therapy can utilize a single dose form containing the vasopeptidase inhibitor or inhibitors or a pharmaceutically acceptable salt thereof, and the other pharmaceutically active agent, co-administration of separate doses of each active agent, or administration of separate doses of each active agent according to a staggered schedule.

### DETAILED DESCRIPTION OF THE INVENTION

In accordance with the present invention, a method is provided for inhibiting loss of cognitive functions such as memory, attention span, concentration and the ability to learn. A method is also provided for preventing and/or treating dementia, including both senile dementia of the Alzheimer's type and vascular dementia. Both senile dementia of the Alzheimer's type and vascular dementia are related to the presence of atherosclerotic disease and lipohyalinosis. The relationship between hypertension and atherogenic factors are discussed by Skoog et al., “15-year longitudinal study of blood pressure and dementia”, Lancet (1996), Vol. 347, p. 1141-1145, Hofman et al., “Atherosclerosis, apolipoprotein E, and pevalence of dementia and Alzheimer's disease in the Rotterdam study”, Lancet (1997), Vol. 349, p. 151-151, and Lis et al., “Vascular dementia, hypertension, and the brain”, Neurological Research, Vol. 19, p. 471-480 (1997).

This invention is directed to the use of one or more vasopeptidase inhibitors to treat and/or slow the progression of cognitive dysfunction and to treat and/or prevent dementia, including senile dementia of the Alzheimer's type and vascular dementia. Preferred vasopeptidase inhibitors for this use are omapatrilat or a pharmaceutically acceptable salt thereof, or the combination of omapatrilat or a pharmaceutically acceptable salt thereof and a diuretic, preferably hydrochlorothiaizide, and gemopatrilat or a pharmaceutically acceptable salt thereof, or the combination of gemopatrilat or a pharmaceutically acceptable salt thereof and a diuretic, preferably, hydrochlorothiazide. Omapatrilat is the most preferred vasopeptidase inhibitor for this use.

The vasopeptidase inhibitor can be administered to a patient suffering from cognitive dysfunction and/or dementia. It can also be administered to a patient suffering from hypertension to prevent dementia. In either circumstance, the vasopeptidase inhibitor or a pharmaceutically acceptable salt thereof or the combination with a diuretic can be administered at from about 2.5 mg to about 240 mg per 24 hours, preferably from about 10 mg to about 80 mg per 24 hours. The vasopeptidase inhibitor can be administered in one or more doses over the 24 hour period to provide the total amount of active agent within the above range. If more than one dose is administered per 24 hours, the doses may be equal or may be varied in strength. Of course, the amount of active agent employed will be adjusted by the physician according to the type and severity of the hypertension, cognitive dysfunction and/or dementia. Also, if a combination of vasopeptidase inhibitors is employed, then one or both of the inhibitors may be administered in a lesser amount provided that the total combination of active agents administered is within the above range.

The vasopeptidase inhibitor is preferably administered orally in tablet or capsule form. However, other methods of administration may also be utilized including sublingually, bucally, parenterally such as by subcutaneous, intraveneous, or intramuscular injection or infusion techniques, nasally such as by inhalation spray, topically such as in the form of a cream or ointment, transdermally as in the form of a patch that is applied to the skin, or rectally such as in the form of suppositories. The various dosage formulations contain in addition to the vasopeptidase inhibitor conventional pharmaceutically acceptable vehicles, stabilizers, preservatives, lubricants, diluents, and other conventional ingredients. The formulation may be administered for immediate release or extended release.

Another aspect of this invention is the treatment of cognitive dysfunction and/or dementia with one or more vasopeptidase inhibitors, as defined above, in combination with another class of pharmaceutically active agents known to be useful in the treatment of cognitive dysfunction and/or dementia. In such combination therapies, the amount of vasopeptidase inhibitor may be less than the amount employed in the monotherapy described above.

The vasopeptidase inhibitor and the other pharmaceutically active agent or agents may be formulated as a single dosage form, may be co-administered from separate dosage forms, or may be administered from separate dosage forms according to a staggered schedule.

The term pharmaceutically acceptable salt includes alkali metal salts such as sodium and potassium, alkaline earth metal salts such as calcium and magnesium, salts derived from amino acids such as arginine, lysine, etc. and salts derived from amines such as alkylamines, e.g. t-butylamine, t-amylamine, etc., substituted alkylamines, e.g. benzylamine, dialkylamines, substituted dialkylamines, e.g. N-methyl glucamine, trialkylamines, substituted trialkylamines, and quaternary ammonium salts.

Suitable diuretics include the thiazide type such as hydrochlorothiazide, which is preferred, bendroflumethiaziade, etc.

What is claimed is:

1. A method of treating and/or slowing the progression of cognitive dysfunction and treating and/or preventing dementia comprising administering an effective amount of a vasopeptidase inhibitor.

2. A method of claim 1 wherein said vasopeptidase inhibitor is selected from the group consisting of omapatrilat, a pharmaceutically acceptable salt of omapatrilat, the combination of omapatrilat or a pharmaceutically acceptable salt thereof and a diuretic, gemopatrilat, a pharmaceutically acceptable salt of gemopatrilat, the combination of gemopatrilat or a pharmaceutically acceptable salt thereof and a diuretic, and mixtures thereof.

3. The method of claim 2 wherein said vasopeptidase inhibitor is omapatrilat.

4. The method of claim 1 wherein the condition being treated is cognitive dysfunction.

5. The method of claim 1 wherein the condition being treated is senile dementia of the Alzheimer's type or vascular dementia.

6. A method of treating and/or slowing the progression of cognitive dysfunction and treating and/or preventing dementia comprising administering an effective amount of a vasopeptidase inhibitor together with another pharmaceutically active agent known to be useful in the treatment of cognitive dysfunction and/or dementia.

7. A method of claim 6 wherein said vasopeptidase inhibitor is selected from the group consisting of omapatrilat, a pharmaceutically acceptable salt of omapatrilat, the combination of omapatrilat or a pharmaceutically acceptable salt thereof and a diuretic, gemopatrilat, a pharmaceutically acceptable salt of gemopatrilat, the combination of gemopatrilat or a pharmaceutically acceptable salt thereof and a diurectic, and mixture thereof.

8. The method of claim 7 wherein said vasopeptidase inhibitor is omapatrilat.

9. A method of claim 6 wherein said other pharmaceutically active agent is co-administered with said vasopeptidase inhibitor.

10. A method of claim 6 wherein said other pharmaceutically active agent is administered separately from said vasopeptidase inhibitor.

11. A method of claim 6 wherein the condition being treated and/or slowed is cognitive dysfunction.

12. A method of claim 6 wherein the condition being treated and/or prevented is senile dementia of the Alzheimer's type or vascular dementia.
